(19) United States
(12) Patent Application Publication
(10) Pub. No.: US 2017/0340314 A1
(43) Pub. Date: Nov. 30, 2017

(54) METHOD AND APPARATUS FOR GENERATING FUSED ULTRASONIC IMAGE

(71) Applicant: General Electric Company, Schenectady, NY (US)

(72) Inventors: Zhiqiang Jiang, Wuxi (CN); Gang Liu, Wuxi (CN); Rong Lu, Wuxi (CN)

(21) Appl. No.: 15/602,790

(22) Filed: May 23, 2017

(30) Foreign Application Priority Data
May 27, 2016 (CN) 201610362609.2

(51) Int. Cl. A61B 8/08; A61B 8/14; A61B 8/06

(52) U.S. Cl. CPC A61B 8/5246; A61B 8/5207; A61B 8/06; A61B 8/14; A61B 8/0891

(57) ABSTRACT

The present invention relates to a method, apparatus for generating a fused ultrasonic image and an ultrasound machine. The method comprises: acquiring an ultrasonic image of PW mode; acquiring an ultrasonic image of M mode; and superimposing the ultrasonic image of PW mode to a corresponding position of the ultrasonic image of M mode to generate a fused ultrasonic image.

### FIELD OF THE INVENTION

The invention relates to the technical field of ultrasound, particularly to a method and apparatus for generating a fused ultrasonic image.

### BACKGROUND OF THE INVENTION

Ultrasound scan may be performed in a plurality of different modes to acquire many types of different information. For example, an image acquired by ultrasound scan in an M mode may reflect movement information of a scanned object (e.g., heart, blood vessel or the like) varying over time, and an image acquired by ultrasound scan in a PW (Pulsed Doppler) mode may reflect flow velocity information of a scanned object (e.g., blood or the like) varying over time.

Since some information reflected in an ultrasonic image of M mode and an ultrasonic image of PW mode may have a correlation with each other, a doctor wishes that comparative analysis can be performed for the two.

However, in the prior art, the ultrasonic image of M mode and the ultrasonic image of PW mode can only be presented to an ultrasound operator respectively, the two can even not be presented at the same time and even less be fused together.

Therefore, these is a need to provide a method, apparatus for generating a fused ultrasonic image and a corresponding ultrasound machine, which can fuse an ultrasonic image of M mode and an ultrasonic image of PW mode to be presented to a doctor at the same time.

### BRIEF DESCRIPTION OF THE INVENTION

An objective of the present invention is to provide a method, apparatus for generating a fused ultrasonic image and a corresponding ultrasound machine in order to solve the above technical problems existing in the prior art.

One embodiment of the present invention provides a method for generating a fused ultrasonic image, comprising: acquiring an ultrasonic image of PW mode; acquiring an ultrasonic image of M mode; and superimposing the ultrasonic image of PW mode to a corresponding position of the ultrasonic image of M mode to generate a fused ultrasonic image.

Another embodiment of the present invention provides an apparatus for generating a fused ultrasonic image, comprising: a first image acquisition module for acquiring an ultrasonic image of PW mode; a second image acquisition module for acquiring an ultrasonic image of M mode; and an image fusion module for superimposing the ultrasonic image of PW mode to a corresponding position of the ultrasonic image of M mode to generate a fused ultrasonic image.

### BRIEF DESCRIPTION OF THE DRAWINGS

The present invention can be better understood in light of the following description of embodiments of the present invention with reference to the accompanying drawings, in which:

FIG. 1 illustrates a schematic flow chart of one embodiment of a method for generating a fused ultrasonic image according to the present invention;

FIG. 2 illustrates a schematic flow chart of one embodiment of generating a three-dimensional (3D) ultrasonic image of PW mode in the process of generating a fused ultrasonic image according to the present invention;

FIG. 3 illustrates a schematic diagram of a sub-range division for scan in a PW mode;

FIG. 4 illustrates a diagram of a 3D ultrasonic image of PW mode before being cut out;

FIG. 5 illustrates a schematic flow chart of another embodiment of a method for generating a fused ultrasonic image according to the present invention;

FIG. 6 illustrates a diagram of a 3D ultrasonic image of PW mode after being cut out;

FIG. 7A is a diagram of an ultrasonic image of M mode before being cut out;

FIG. 7B is a diagram of an ultrasonic image of M mode after being cut out; and

FIG. 8 illustrates a schematic block diagram of one embodiment of an apparatus for generating a fused ultrasonic image according to the present invention.

### DETAILED DESCRIPTION OF THE INVENTION

Hereafter, a detailed description will be given for preferred embodiments of the present disclosure. It should be pointed out that in the detailed description of the embodiments, for simplicity and conciseness, it is impossible for the Description to describe all the features of the practical embodiments in details. It should be understood that in the process of a practical implementation of any embodiment, just as in the process of an engineering project or a designing project, in order to achieve a specific goal of the developer and in order to satisfy some system-related or business-related constraints, a variety of decisions will usually be made, which will also be varied from one embodiment to another. In addition, it can also be understood that although the effort made in such developing process may be complex and time-consuming, some variations such as design, manufacture and production on the basis of the technical contents disclosed in the disclosure are just customary technical means in the art for those of ordinary skilled in the art relating to the contents disclosed in the present invention, which should not be regarded as insufficient disclosure of the present invention.

Unless defined otherwise, all the technical or scientific terms used in the Claims and the Description should have the same meanings as commonly understood by one of ordinary skilled in the art to which the present disclosure belongs. The terms “first”, “second” and the like in the Description and the Claims of the present utility model do not mean any sequential order, number or importance, but are only used for distinguishing different components. The terms “a”, “an” and the like do not denote a limitation of quantity, but denote the existence of at least one. The terms “comprises”, “comprising”, “includes”, “including” and the like mean that the element or object in front of the “comprises”, “comprising”, “includes” and “including” covers the elements or objects and their equivalents illustrated following the “comprises”, “comprising”, “includes” and “including”, but do not exclude other elements or objects. The term “coupled” or “connected” or the like is not limited to being connected physically or mechanically, nor limited to being connected directly or indirectly.

In order to make the purpose, the technical solutions and the advantages of the invention more apparent, the technical solutions of the present invention will be set forth clearly and fully in the following by combining with specific embodiments of the invention and the corresponding accompanying drawings. Obviously, the described embodiments are merely part—not all—of the embodiments in the present invention. In view of the embodiments in the present invention, other embodiments made by one of ordinary skilled in the art without inventive work all fall within the scope of protection of the invention.

According to an embodiment of the present invention, a method for generating a fused ultrasonic image is provided.

The fused ultrasonic image as described herein refers to an image obtained by fusing ultrasonic images together that are obtained by at least two different ultrasound scan modes.

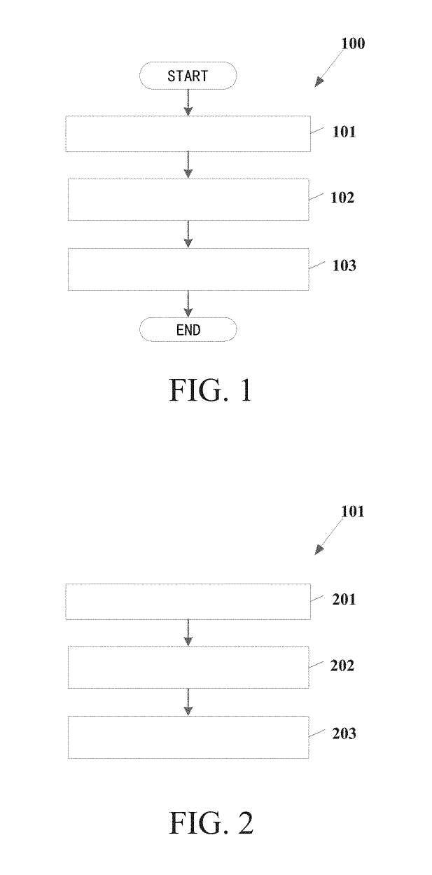

With reference to FIG. 1, FIG. 1 illustrates a schematic flow chart of one embodiment 100 of a method for generating a fused ultrasonic image according to the present invention. The embodiment 100 may comprise the following steps 101-103.

As shown in FIG. 1, in Step 101, an ultrasonic image of PW mode is acquired.

In one embodiment of the present invention, a 3D ultrasonic image of PM mode may be acquired. With reference to FIG. 2, FIG. 2 illustrates steps of a schematic diagram of one embodiment of generating a 3D ultrasonic image of PW mode. In the embodiment as shown in FIG. 2, Step 101 may further comprise the following sub-steps 201-203.

In Sub-step 201, a range for PW scan is divided into a plurality of sub-ranges.

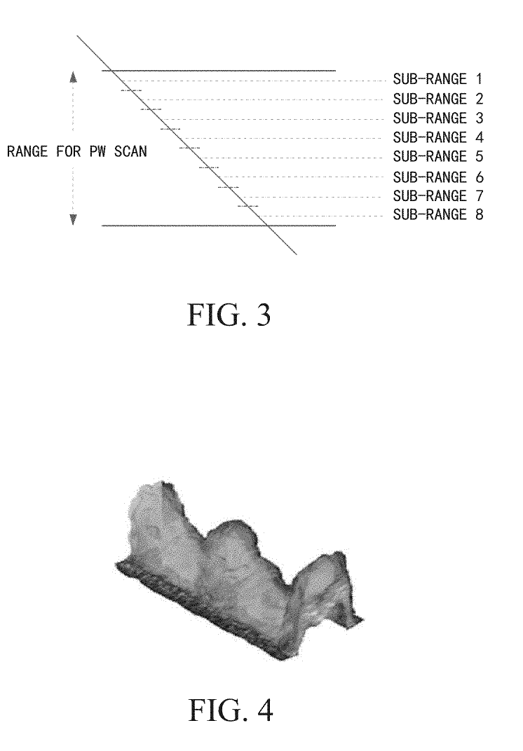

Take PW scan performed on a blood vessel as an example, as shown in FIG. 3, a range for PW scan on the blood vessel may be divided equally or unequally into N sub-ranges, in which N may be preset. In FIG. 3, the range is divided into 8 sub-ranges.

In Sub-step 202, PW scan is performed on each of a plurality of sub-ranges to acquire multiple groups of PW sub-data.

For each of the N sub-ranges divided in Sub-step 201, a group of corresponding PW sub-data may be acquired. Thereby, there are altogether N groups of PW sub-data.

In one embodiment of the present invention, a spatial resolution for PW scan may be changed by the way of adjusting a decimation rate so as to acquire PW scan data of N sub-ranges.

In Sub-step 203, the multiple groups of PW sub-data are superimposed to generate a 3D ultrasonic image of PW mode.

For the N groups of PW sub-data acquired in Sub-step 202, imaging may be performed on each group of sub-data so as to obtain N ultrasonic sub-images of PW mode. Then, after these N ultrasonic sub-images of PW mode are superimposed together, a 3D ultrasonic image of PW mode as shown in FIG. 4 may just be obtained.

In Step 102, an ultrasonic image of M mode is acquired.

In one embodiment of the present invention, the ultrasonic image of M mode may be acquired in an M mode directly. In another embodiment of the present invention, a scanning line of an M mode may also be acquired by a two-dimensional (2D) image of B mode so as to obtain the ultrasonic image of M mode. In yet another embodiment of the present invention, data required for generating the ultrasonic image of M mode may also be obtained with a scanning line of a PW mode.

In Step 103, the ultrasonic image of PW mode is superimposed to a corresponding position of the ultrasonic image of M mode to generate a fused ultrasonic image.

Take a scanned object, which is a blood vessel and blood therein, as an example, since the ultrasonic image of PW mode reflects a flow rate at which the blood varies over time, the ultrasonic image of PW mode may be superimposed to a position inside the blood vessel on the ultrasonic image of M mode to generate the fused ultrasonic image. For example, the image of an area inside the blood vessel on the ultrasonic image of M mode may be covered or deleted, and then after the ultrasonic image of PW mode is superimposed to said area, the fused ultrasonic image can just be obtained.

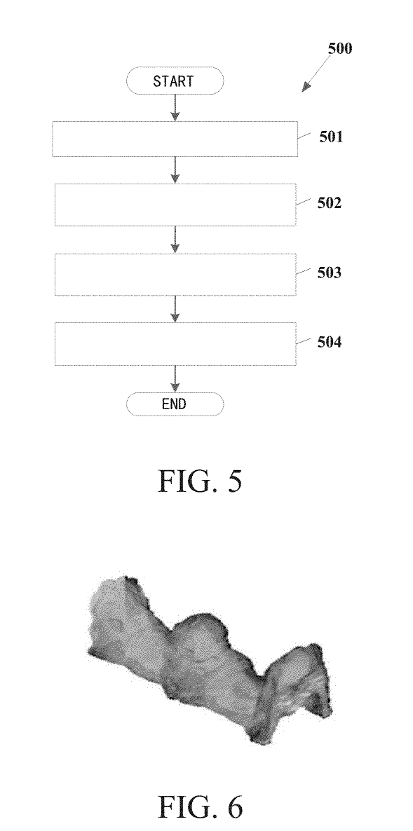

With reference to FIG. 5, FIG. 5 illustrates a schematic flow chart of another embodiment 500 of a method for generating a fused ultrasonic image according to the present invention. The embodiment 500 may comprise the following steps 501-504.

In Step 501, a PW gate is adjusted according to a regular pattern in which a scanned object varies over time in a PW mode.

In the process of PW scan, a position of an ultrasound probe may be moved. Moreover, take a blood vessel as an example, its shape would vary over time due to diastole and systole. When the blood vessel is systolic, its lumen will become narrower; and when the blood vessel is diastolic, its lumen will become wider. Therefore, in the process of the PW scan, a size of the PW gate may be adjusted according to a regular pattern in which the scanned object varies over time.

In one embodiment of the present invention, the size of the PW gate may be determined by recognizing an area of a lumen of the blood vessel on the PW ultrasonic image. In another embodiment of the present invention, the size of the lumen of the blood vessel may also be determined from a strength of a PW echo signal because an envelop of the echo signal will obviously change in strength at a position of a junction of the blood and a wall of the blood vessel. Of course, the above two methods may also be combined with each other.

Step 502 is similar to the above Step 101, but may further cut out the ultrasonic image of PW mode by utilizing the PW gate that varies in real time obtained in Step 501. FIG. 6 illustrates a 3D ultrasonic image of PW mode after being cut out, in which an image that doesn't belong to the blood stream portion has been removed from the cut-out ultrasonic image of PW mode as can be seen from comparison with FIG. 4.

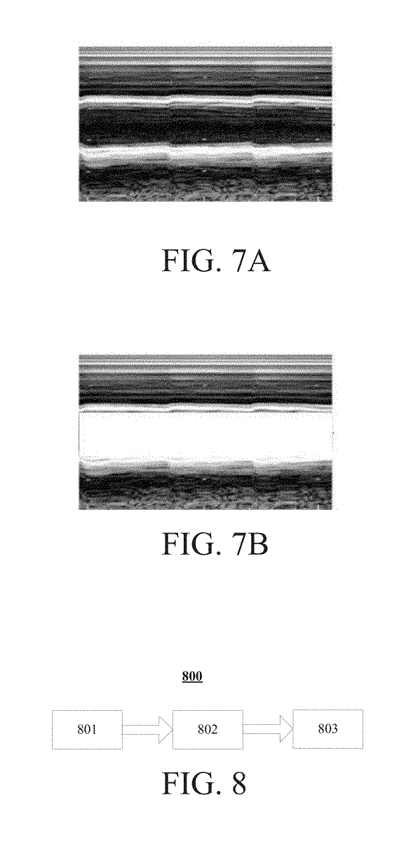

Step 503 is similar to the above Step 102, but may further cut out the ultrasonic image of M mode by utilizing the PW gate that varies in real time obtained in Step 501. FIG. 7A illustrates an ultrasonic image of M mode before being cut out, and FIG. 7B illustrates an ultrasonic image of M mode after being cut out. It can be seen that an image located within a blood vessel lumen has been removed from the cut-out ultrasonic image of M mode.

Step 504 is similar to the above Step 103, and the 3D ultrasonic image of PW mode after being cut out in Step 502 may be superimposed to a vacant position of the ultrasonic image of M mode after being cut out in Step 503 to obtain a fused ultrasonic image.

So far, the method for generating a fused ultrasonic image according to the embodiments of the present invention has been described. According to the method of the present invention, the ultrasonic image of PW mode and the ultrasonic image of M mode can be fused together so as to be presented to a doctor, such that the doctor can more easily analyze the related information such as a relationship between a flow velocity and a movement of an organ tissue or the like. Moreover, the ultrasonic image of PW mode presented in a 3D form also allows the doctor to selectively view a sub-image and/or a sub-audio generated by a part of sub-data therein, enhancing operability of the doctor.

Similar to the method, the present invention also provides a corresponding apparatus.

FIG. 8 illustrates a schematic block diagram of one embodiment of an apparatus for generating a fused ultrasonic image according to the present invention.

As shown in FIG. 8, an apparatus 800 may include: a first image acquisition module 801 for acquiring an ultrasonic image of PW mode; a second image acquisition module 802 for acquiring an ultrasonic image of M mode; and an image fusion module 803 for superimposing the ultrasonic image of PW mode to a corresponding position of the ultrasonic image of M mode to generate a fused ultrasonic image.

In one embodiment of the present invention, the apparatus 800 may also include: a PW gate adjustment module for adjusting a PW gate according to a regular pattern in which a scanned object varies over time in a PW mode.

In one embodiment of the present invention, the first image acquisition module 801 may further include: a first image cutting out module for cutting out the ultrasonic image of PW mode according to the PW gate.

In one embodiment of the present invention, the first image acquisition module 801 may further include: a sub-range division module for dividing a range for PW scan into a plurality of sub-ranges; a sub-data acquisition module for performing PW scan on each of the plurality of sub-ranges to acquire multiple groups of PW sub-data; and a 3D image generation module for superimposing the multiple groups of PW sub-data to generate a 3D ultrasonic image of PW mode.

In one embodiment of the present invention, the second image acquisition module 802 may further include: a second image cutting out module for cutting out the ultrasonic image of M mode according to the PW gate.

In one embodiment of the present invention, the image fusion module 803 may further include: a superimposition module for superimposing a cut-out ultrasonic image of PW mode onto a cut-out ultrasonic image of M mode.

So far, the apparatus for generating a fused ultrasonic image according to the embodiments of the present invention has been described. Similar to the above method, the apparatus according to the present invention can fuse the ultrasonic image of PW mode and the ultrasonic image of M mode together to be presented to a doctor, such that the doctor can more easily analyze the related information such as a relationship between a flow velocity and a movement of an organ tissue or the like. Moreover, the ultrasonic image of PW mode presented in a 3D form also allows the doctor to selectively view a sub-image and/or a sub-audio generated by a part of sub-data therein, enhancing operability of the doctor.

The above descriptions are merely embodiments of the invention and are not intended to restrict the scope of the invention. All kinds of variations and modifications could be made to the present invention to those skilled in the art. Any modifications, alternatives and improvements made within the spirit and principles of the present invention shall fall within the scope of the appended claims.

We claim:

1. A method for generating a fused ultrasonic image, comprising:
acquiring an ultrasonic image of PW mode;
acquiring an ultrasonic image of M mode; and
superimposing said ultrasonic image of PW mode to a corresponding position of said ultrasonic image of M mode to generate a fused ultrasonic image.

2. The method according to claim 1, further comprising:
adjusting a PW gate according to a regular pattern in which a scanned object varies over time in a PW mode.

3. The method according to claim 2, wherein said step of acquiring an ultrasonic image of PW mode further comprises:
cutting out said ultrasonic image of PW mode according to said PW gate.

4. The method according to claim 3, wherein said step of acquiring an ultrasonic image of PW mode further comprises:
dividing a range for PW scan into a plurality of sub-ranges;
performing PW scan on each of said plurality of sub-ranges to acquire multiple groups of PW sub-data; and
superimposing said multiple groups of PW sub-data to generate a 3D ultrasonic image of PW mode.

5. The method according to claim 2, wherein said step of acquiring an ultrasonic image of M mode further comprises
cutting out said ultrasonic image of M mode according to said PW gate.

6. The method according to claim 5, wherein said step of superimposing said ultrasonic image of PW mode to a corresponding position of said ultrasonic image of M mode to generate a fused ultrasonic image further comprises:
superimposing a cut-out ultrasonic image of PW mode onto a cut-out ultrasonic image of M mode.

7. An apparatus for generating a fused ultrasonic image, comprising:
a first image acquisition module for acquiring an ultrasonic image of PW mode;
a second image acquisition module for acquiring an ultrasonic image of M mode; and
an image fusion module for superimposing said ultrasonic image of PW mode to a corresponding position of said ultrasonic image of M mode to generate a fused ultrasonic image.

8. The apparatus according to claim 7, further comprising:
a PW gate adjustment module for adjusting a PW gate according to a regular pattern in which a scanned object varies over time in a PW mode.

9. The apparatus according to claim 8, wherein said first image acquisition module further comprises:
a first image cutting out module for cutting out said ultrasonic image of PW mode according to said PW gate.

10. The apparatus according to claim 9, wherein said first image acquisition module further comprises:
a sub-range division module for dividing a range for PW scan into a plurality of sub-ranges;
a sub-data acquisition module for performing PW scan on each of said plurality of sub-ranges to acquire multiple groups of PW sub-data; and
a 3D image generation module for superimposing said multiple groups of PW sub-data to generate a 3D ultrasonic image of PW mode.

11. The apparatus according to claim 8, wherein said second image acquisition module further comprises:
a second image cutting out module for cutting out said ultrasonic image of M mode according to said PW gate.

12. The apparatus according to claim 11, wherein said image fusion module further comprises:
a superimposition module for superimposing a cut-out ultrasonic image of PW mode onto a cut-out ultrasonic image of M mode.
